(19) United States
(12) Patent Application Publication
(10) Pub. No.: US 2023/0201031 A1
(43) Pub. Date: Jun. 29, 2023

(54) DISSOLVABLE MEDICAL DEVICE FOR PROMOTING HEALING OF WOUNDS

(71) Applicant: Alcon Inc., Fribourg (CH)

(72) Inventors: Howard Allen Ketelson, Dallas, TX (US); Rekha Rangarajan, Fort Worth, TX (US)

(21) Appl. No.: 18/145,437

(22) Filed: Dec. 22, 2022

Related U.S. Application Data
(60) Provisional application No. 63/265,971, filed on Dec. 23, 2021.

(51) Int. Cl. A61F 9/007; A61F 2/14; A61F 9/00; A61L 17/10; A61L 31/14

(52) U.S. Cl. CPC A61F 9/007; A61F 2/142; A61F 9/0017; A61L 17/10; A61L 31/148

(57) ABSTRACT

A dissolvable medical device for placing on the outer exposed surface of the eye to heal the corneal wound comprising of: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers, wherein the dissolved polymeric film is not impeding visualization of ocular tissue while maintaining a protective film on outer surface of the eye.

### FIELD OF THE DISCLOSURE

The present disclosure generally relates to a dissolvable medical device for placing on the outer exposed surface of the eye to heal the corneal wound created post-surgery.

### BACKGROUND

Ophthalmic operations such as glaucoma surgery, vision correction laser surgeries like LASIK or PRK for treating refraction defects and cataract surgery are involved procedures performed by surgeons making an incision in the eye with cutting instruments. At the incision site the cutting edge of these instruments unavoidably damages several layers of cells on either side of the point of entry. This impairs the ability of the surgical wound to heal without resulting scar tissue. For glaucoma surgery, incisional surgery (also called filtering surgery) involves creating a drainage hole with the use of a small surgical tool. This new opening allows the intraocular fluid to bypass the clogged drainage canals and flow out of this new, artificial drainage canal. Treatment of refraction defects such as spherical ametropias (myopia and hypermetropia) and cylindrical ametropias (astigmatism) has progressed in recent years beyond application of prosthetic devices designed to correct the defects, including eyeglasses and contact lenses. Surgical procedures are now commonly performed to achieve a measure of permanence in the correction of refraction defects. For example, radial keratotomy (RK) is a surgical procedure which corrects the shape of the eye by placing radial incisions in the periphery of the eye to change the curvature of the cornea. More recent advances include the treatment known as photorefractive keratectomy (PRK). In PRK, a laser is used to reshape the surface of the cornea by ablating a portion of the outer layers of the cornea. The preferred laser for PRK is the excimer laser which emits radiation with a wavelength of 193 nm. At this wavelength, photoablation results without thermal damage to deeper cells. PRK differs from RK and other surgical procedures by not altering the inner layers of the cornea (corneal endothelium); in PRK, only the outer layers of cells of the cornea are removed. On the other hand, in other laser procedures such as laser in situ keratomileusis (LASIK), ablation of stromal tissue is performed. Cataract surgery is a procedure to remove the lens of your eye and, in most cases, replace it with an artificial lens. Using an ultrasound probe to break up the lens for removal. During a procedure called phacoemulsification, surgeon makes a tiny incision in the front of your eye (cornea) and inserts a needle-thin probe into the lens substance where the cataract has formed. Then surgeon uses the probe, which transmits ultrasound waves, to break up (emulsify) the cataract and suction out the fragments. The very back of your lens (the lens capsule) is left intact to serve as a place for the artificial lens to rest. Stitches may be used to close the tiny incision in your cornea at the completion of the procedure. Collagen shields are prescribed for patients post-surgery as a way to promote healing of the ocular surface.

Graft-versus-host disease in the eye (Ocular GVHD) involves both cell-mediated and humoral immunity that leads to infiltration and inflammation of the lacrimal gland, conjunctiva and ocular surface. The inflammation can eventually cause a decrease in the density of conjunctival goblet cells as well as scarring of the lacrimal gland and conjunctiva. Current treatment for ocular GVHD includes topical cyclosporine 0.05% (Restasis, Allergan). Topical loteprednol etabonate 0.5% (Lotemax, Bausch and Lomb) has been shown to be safe and efficacious in treatment of inflammatory ocular disorders.

Exposure to topically administered aqueous formulations is often driven by the short retention time of the formulation on the ocular surface. Typical aqueous formulations for ocular use may be diluted or washed from the ocular surface within minutes, introduce variability in the usage, or result in less accurate and precise dosages administered to the eye. Accordingly, there is a need to reduce treatment burden and improve compliance.

### SUMMARY

The invention, in one aspect, provides a dissolvable medical device for placing on the outer exposed surface of the eye to heal the corneal wound comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers, wherein the dissolved polymeric film is not impeding visualization of ocular tissue while maintaining a protective film on outer surface of the eye.

The invention, in another aspect, provides a method for healing a corneal wound comprising: (a) identifying a subject in need of healing for a corneal wound selected from the group consisting of glaucoma surgery wound, cataract surgery wound, vision correction laser surgery wound and GVHD induced ocular surface damage, and (b) placing a dissolvable medical device to the outer exposed surface of the eye to heal the corneal wound, wherein the polymeric film dissolves between 15 minutes to 120 minutes to release the mucoadhesive polymers, wherein the dissolved dissolvable medical device is not impeding visualization of ocular tissue while maintaining a protective film on outer surface of the eye.

### BRIEF DESCRIPTION OF THE DRAWINGS

For a more complete understanding of this disclosure, reference is now made to the following description, taken in conjunction with the accompanying drawings, in which:

FIGS. 1A-1C illustrate the compatibility of the polymeric film during simulated surgical procedures.

FIGS. 2A-2B illustrate the compatibility of the polymeric film on visualization during a simulated surgery.

FIG. 3 illustrates the effect of the dissolvable medical device (corneal shield) and a topical steroid on healing of the damage corneal surface according to the percent of Keratopathy still present.

FIG. 4 illustrates the effect of the dissolvable medical device (corneal shield) and the topical steroid on healing of the damage corneal according to the Fluorescein Index Change test.

### DETAILED DESCRIPTION

Reference now will be made in detail to the embodiments of the invention, one or more examples of which are set forth below. Each example is provided by way of explanation of the invention, and is not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. As employed throughout the disclosure, the following terms, unless otherwise indicated, shall be understood to have the following meanings.

The invention, in one aspect, provides a dissolvable medical device for placing on the outer exposed surface of the eye to heal the corneal wound comprising of: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive, wherein the dissolved polymeric film is not impeding visualization of ocular tissue while maintaining a protective film on outer surface of the eye.

It has been discovered that a dissolvable medical device comprising a polymeric film which comprises one or more mucoadhesive polymers is well suitable for placing on the outer exposed surface of the eye to heal the corneal wound post-surgery. The polymeric film of the invention is characterized by having a dimension substantially covering a cornea when applied to an eye and characterized by dissolving between 15 minutes and 120 minutes to release the mucoadhesive polymers after the film has been applied to the eye. In addition, the dissolved polymeric film is not impeding visualization during its presence on outer surface of the eye.

The dissolvable medical device as an ophthalmic surgical device can offer some advantages over a collagen shield. First, it is much easier to dissolve a dissolvable medical device of the present invention than to dissolve a collagen shield. The currently commercially available collagen shields have dissolution times of 12, 24, and 72 hours. The amount of crosslinking induced in the collagen shield by UV irradiation during manufacture determines the length of time the shield will remain intact and on the eye. In contrast, the polymeric film of the present invention has dissolution times of between 15 minutes to 120 minutes. Second, the dissolvable medical device of the present invention is not impeding visualization during maintaining a protective surface on the outer eye. In contrast, upon contact with enzymes that are present in the tears on the eye, the collagen shield will begin to swell and become cloudy, resulting in a loss of transparency. The loss of transparency of the collagen shields shortly after being placed on the eye is the biggest problem with the collagen shields. Because the cloudiness interfere during the possible following surgical procedures and impeded visualization after surgical procedures. Third, the dissolvable medical device of the present invention provides an effective healing the corneal wound.

The biomaterial for forming a dissolvable medical device according to embodiments of the present disclosure may be comprised of one or more polymers that are biocompatible with the ocular surface and tear film. Polymers that may be used in dissolvable medical device according to embodiments of the present disclosure include, but are not limited to, hyaluronic acid (in acid or salt form), hydroxypropylmethylcellulose (HPMC), methylcellulose, tamarind seed polysaccharide (TSP), Galactomannans, for examples; guar and derivatives thereof such as hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and/or combinations thereof.

The preferred biocompatible polymers are hyaluronic acid, guar and derivatives and/or combinations thereof. Hyaluronic acid is an unsulphated glycosaminoglycan composed of repeating disaccharide units of N-acetylglucosamine (GlcNAc) and glucuronic acid (GlcUA) linked together by alternating beta-1,4 andbeta-1,3 glycosidic bonds. Hyaluronic acid is also known as hyaluronan, hyaluronate, or HA. As used herein, the term hyaluronic acid also includes salt forms of hyaluronic acid such as sodium hyaluronate. A preferred hyaluronic acid is sodium hyaluronate. The weight average molecular weight of the hyaluronic acid used in insert of the present invention may vary, but is typically weight average of 0.75 to 5.0 M Daltons. In one embodiment, the HA has a weight average molecular weight of 0.75 to 4 M Daltons. In another embodiment, the HA has a weight average molecular weight of 1 to 4 M Daltons.

The galactomannans of the present invention may be obtained from numerous sources. Such sources include from fenugreek gum, guar gum, locust bean gum and tara gum. Additionally, the galactomannans may also be obtained by classical synthetic routes or may be obtained by chemical modification of naturally occurring galactomannans. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with. Alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 are most preferred. Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic variations to the galactomannans, such as those containing alkoxy and alkyl (C1-C6) groups are particularly preferred when a soft gel is desired (e.g., hydroxylpropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired, Preferred galactomannans of the present invention are guar and hydroxypropyl guar. Hydroxypropyl guar is particularly preferred. The weight average molecular weight of the Hydroxypropyl guar in the dissolvable medical device of the present invention may vary, but is typically but is typically 1 to 5M Daltons. In one embodiment, the Hydroxypropyl guar has a weight average molecular weight of 2 to 4 MDaltons. In another embodiment, the Hydroxypropyl guar has a weight average molecular weight of 3 to 4 M Daltons.

Polymers used in dissolvable medical devices according to embodiments of the present disclosure should be non-toxic and able to solubilize in eye fluids to ensure that the insert is eventually cleared from the eye, generally within 15 to 120-minute time frame. It should be appreciated that the polymer(s) selected should be mucoadhesive. It also should be appreciated that one or more polymers may be blended according to embodiments of the present disclosure. For example, in an embodiment of the present disclosure, hyaluronic acid (HA) may be blended with tamarind seed polysaccharide (TSP) because TSP has been shown to increase residence time of HA in aggregate blends and the blend has desired film mechanical and lubrication properties. In other embodiments of the present disclosure, as described in further detail below, hyaluronic acid may be combined with HP guar.

In some embodiments of the present disclosure, the preferred biocompatible polymers also include polyvinyl pyrrolidine (PVP). PVP is also a mucoadhesive polymer. The weight average molecular weight of the PVP in the polymeric film of the present invention may vary, but is typically 4,000 Dalton to 3 M Daltons. In one embodiment, the PVP has a weight average molecular weight of 40 K Daltons to 2 M Daltons. In another embodiment, the PVP has a weight average molecular weight of 0.5 M Daltons to 2 M Daltons.

In some embodiments of the present disclosure, a softener and/or plasticizer may be added to the one or more polymers to facilitate fabrication of a softer, malleable delivery system and also provide improved comfort in covering the cornea. A plasticizer may soften the material to provide for desirable dissolution rates. It should be appreciated softeners and/or plasticizers may be low or high-molecular weight compounds, including not limited to, polyethylene glycol (PEG) and derivatives thereof, water, Vitamin E, and triethyl citrate. The weight average molecular weight of the PEG in the polymeric film of the present invention may vary, but is typically 200 Dalton to 100,000 Daltons. In one embodiment, the PEG has a weight average molecular weight of 200 to 12000 Daltons. In another embodiment, the PEG has a weight average molecular weight of 200 to 6000 Daltons.

In some embodiments, the HP guar is present in an amount of from about 5% to about 60% w/w, preferably 15% to about 50% w/w, more preferably 25% to about 40 w/w by dry weight of the polymeric film. The PVP is present in an amount of from about 1% to about 30% w/w, preferably 5% to about 25% w/w, more preferably 10% to about 20 w/w by dry weight of the polymeric film. The hyaluronic acid (HA) is present in an amount of from about 5% to about 60% w/w, preferably 15% to about 50% w/w, more preferably 25% to about 40 w/w by dry weight of the polymeric film. The PEG is present in an amount of from about 1% to about 30% w/w, preferably 5% to about 25% w/w, more preferably 10% to about 20 w/w by dry weight of the polymeric film. According to the present application, the total amount of ingredients of the polymeric dissolvable medical devices is equal to 100% w/w.

The overall dry weight or mass of the polymeric film may be in the range of about 1 to about 12 mg, or about 2 to about 10 mg, and in particular embodiments may be from about 3 to about 9 mg.

In some embodiments, the polymeric film has a thickness of about 50-300 μm, about 120-250 μm, about 140-200 μm, or preferably about 120 μm.

In some embodiments, the polymeric film has circular shape about 2 mm to 13 mm in diameter or other shapes have the same area corresponding to circular shape about 2 mm to 13 mm in diameter. In still some embodiments, the polymeric film has a contact lens shape and prefers about 11 mm to 13 mm in diameter.

According to the present disclosure, the dissolvable medical device for placing on the outer exposed surface of the eye may beal any kind of the corneal wounds. For example, corneal wounds due to glaucoma surgery, vision correction laser surgeries like LASIK or PRK for treating refraction defects, cataract surgery and Chronic graft-versus-host disease (GVHD) in tissue transplant patients. GVHD often affects the eyes, making it difficult to perform normal, daily functions. It can be the only sign of chronic GVHD, or a signal that chronic GVHD is developing elsewhere in the body, as well. Chronic GVHD of the eye happens when the donor's cells attack the eye conjunctiva and glands. The conjunctiva is the tissue that covers the white part of your eye and the inside of your eyelids. Eye glands make tears that help your eyes stay moist and smooth. Ocular GVHD mimics other immunologically mediated ocular inflammatory diseases. Symptoms that are not unique to this disease entity can include dry eye, foreign body sensation, redness, epiphora, photophobia, blurred vision, eye irritation. Ocular GVHD involves both cell-mediated and humoral immunity that leads to infiltration and inflammation of the lacrimal gland, conjunctiva and ocular surface. The inflammation can eventually cause a decrease in the density of conjunctival goblet cells as well as scarring of the lacrimal gland and conjunctiva.

Current treatment for ocular GVHD includes topical cyclosporine 0.05% (Restasis, Allergan). Topical loteprednol etabonate 0.5% (Lotemax, Bausch and Lomb) has also been shown to be safe and efficacious in treatment of inflammatory ocular disorders. Steroids, like prednisone, are the main treatment for GVHD. Steroids are a kind of medicine called an immunosuppressant. These medicines weaken the new immune system so your new cells don't attack your body.

It is unexpected to discover that the dissolvable medical device of the present disclosure promotes faster healing of the damaged cornea than a topical steroid (loteprednol etabonate 0.5%) within first 24 hours according to the percent of Keratopathy still present and Fluorescein Index Change.

The invention, in another aspect, provides A method for healing a corneal wound comprising: (a) identifying a subject in need of healing for a corneal wound selected from the group consisting of glaucoma surgery wound, cataract surgery wound, vision correction laser surgery wound and GVHD wound.

The invention, in another aspect, provides a method for healing a corneal wound comprising: (a) identifying a subject in need of healing for a corneal wound selected from the group consisting of glaucoma surgery wound, cataract surgery wound, vision correction laser surgery wound and GVHD wound, and (b) placing a dissolvable medical device to the outer exposed surface of the eye to heal the corneal wound, wherein the polymeric film dissolves between 15 minutes to 120 minutes to release the mucoadhesive polymers, wherein the dissolved polymeric film is not impeding visualization of ocular tissue while maintaining a protective film on outer surface of the eye.

The following non-limiting Examples are provided to illustrate embodiments of the invention.

### EXAMPLES

Procedure below on how to manufacture and cast corneal shields. Slight variations in volume casted and drying times based on corneal shield thickness (I to III). Target thickness is −120 microns).

### Example I

Procedure to make 90-120 micron thickness dry films

Part 1: Procedure to prepare 850 g stock solution of the formulation (HA 40%/HPGuar 40%/PVP 10%/PEG 10%) at 0.85 g/100 mL concentration in order to make dry films of 90-120 micron thickness:

850 mL of distilled water were placed in a 1 L Erlenmeyer flask followed by the addition of Hyaluronic acid and PVP. The flask was placed in a sonicator and an overhead mechanical stirrer was set up. The mixture was sonicated and stirrer until a viscous, clear and homogeneous solution was obtained (90±30 minutes). The speed of the mechanical stirrer was adjusted to 450±50 rpm. HPGuar was added and the mixture was sonicated and stirred for another 90±30 minutes. To the clear, viscous and homogeneous solution, PEG 400 was added. The mixture was sonicated and stirred for 30 minutes. The mechanical stirring was then stopped and the sonication was allowed to continue for an additional 30 minutes in order to release all bubbles.

Film Casting Procedure:

For the preparation of the films, a petri dish (150 mm diameter×20 mm height) was filled with 200 g±10 g of the stock solution and placed in the evaporation oven.

The oven is equipped with an exhaust fan to move 110 cfm of air. The temperature inside the oven was controlled at 25±3° C. during the evaporation process.

After 40-48 hours of evaporation, the petri dish was taken out of the oven and placed into a plastic zipped bag overnight. The film was then peeled out and kept in a plastic zipped bag at room temperature.

### Example 2

Procedure to make 140-170 micron thickness dry films

Part 1: Procedure to prepare 800 g stock solution of the formulation (HA 40%/HPGuar 40%/PVP 10%/PEG 10%) at 0.85 g/100 mL concentration in order to make dry films of 140-170 micron thickness:

800 mL of distilled water were placed in a 1 L Erlenmeyer flask followed by the addition of Hyaluronic acid and PVP. The flask was placed in a sonicator and an overhead mechanical stirrer was set up. The mixture was sonicated and stirrer until a viscous, clear and homogeneous solution was obtained (90±30 minutes). The speed of the mechanical stirrer was adjusted to 450±50 rpm. HPGuar was added and the mixture was sonicated and stirred for another 90±30 minutes. To the clear, viscous and homogeneous solution, PEG 400 was added. The mixture was sonicated and stirred for 30 minutes. The mechanical stirring was then stopped and the sonication was allowed to continue for an additional 30 minutes in order to release all bubbles.

Film Casting Procedure:

For the preparation of the films, 1) A 1L beaker was filled with 500 g±10 g of the stock solution and placed in the evaporation oven to reduce the volume to ½ with magnetic stirring. This step takes two days. 2) A petri dish (150 mm diameter×25 mm height) was filled with 270 g±30 g of the concentrated stock solution and placed in the evaporation oven.

The oven is equipped with an exhaust fan to move 110 cfm of air. The temperature inside the oven was controlled at 25±3° C. during the evaporation process.

After 2-3 days of evaporation, the petri dish was taken out of the oven and placed into a plastic zipped bag overnight. The film was then peeled out and kept in a plastic zipped bag at room temperature.

### Example 3

Procedure to make 180-230 micron thickness dry films

Part 1: Procedure to prepare 800 g stock solution of the formulation (HA 40%/HPGuar 40%/PVP 10%/PEG 10%) at 0.85 g/100 mL concentration in order to make dry films of 180-230 micron thickness:

800 mL of distilled water were placed in a 1 L Erlenmeyer flask followed by the addition of Hyaluronic acid and PVP. The flask was placed in a sonicator and an overhead mechanical stirrer was set up. The mixture was sonicated and stirrer until a viscous, clear and homogeneous solution was obtained (90±30 minutes). The speed of the mechanical stirrer was adjusted to 450±50 rpm. HPGuar was added and the mixture was sonicated and stirred for another 90±30 minutes. To the clear, viscous and homogeneous solution, PEG 400 was added. The mixture was sonicated and stirred for 30 minutes. The mechanical stirring was then stopped and the sonication was allowed to continue for an additional 30 minutes in order to release all bubbles.

Film Casting Procedure:

For the preparation of the films, 1) A 1 L beaker was filled with 750 g±20 g of the stock solution and placed in the evaporation oven to reduce the volume to ½ with magnetic stirring. This step takes two-three days. 2) A petri dish (150 mm diameter×25 mm height) was filled with 300 g±30 g of the concentrated stock solution and placed in the evaporation oven.

The oven is equipped with an exhaust fan to move 110 cfm of air. The temperature inside the oven was controlled at 25±3° C. during the evaporation process.

After 3-4 days of evaporation, the petri dish was taken out of the oven and placed into a plastic zipped bag overnight. The film was then peeled out and kept in a plastic zipped bag at room temperature.

### Example 4

In order to determine whether the dissolvable medical device (polymeric film) causes mechanical impediment to standard surgical procedures. The shield was applied on the eye of an anesthetized mouse and allowed to dissolve for 5 minutes. After that, the mouse was euthanized and 3 standard ocular surgical techniques were applied. The first was a corneal incision mimicking that of the clean corneal incisions used in cataract surgery. Then, a small needle was inserted in the incision and fluid was injected in simulating the injection of viscoelastic in multiple ocular surgeries. Lastly, a micro-scissor was used to cut around the cornea using different incisions. During all 3 procedures, no added resistance was noted.

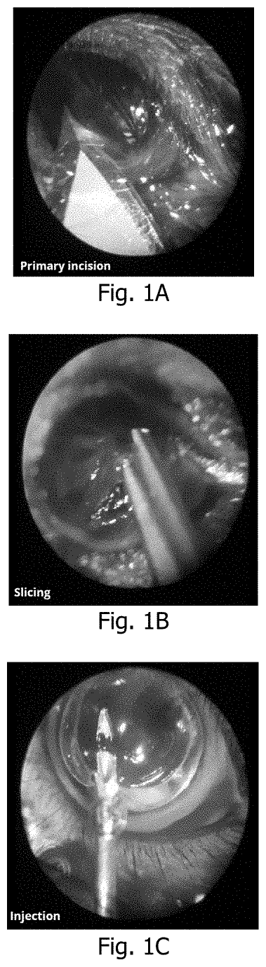

FIGS. 1A, 1B and 1C illustrates that the dissolvable medical device (polymeric film) does not negatively impact surgical procedure such as making incisions, injections and/or slicing of the cornea.

### Example 5

In order to test whether the dissolvable medical device (shield) impedes visualization of the posterior eye, a shield was applied on a euthanized mouse and allowed to dissolve for 5 minutes. After that, a 90D lens was used to visualize the posterior eye. This allowed seeing into the eye and visualizing the optic nerve and the retina.

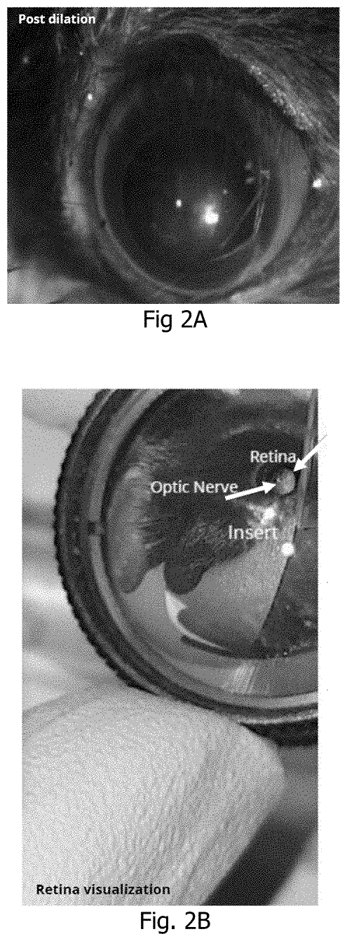

FIGS. 2A and 2B illustrates that the polymeric film does not impair visualization of retina/retinal hallmarks.

### Example 6

In order to test the wound healing properties of the dissolvable medical device (Corneal Shield), a GVHD mouse model was used. C3H mice were transplanted with bone marrow and T-cells from a BL/6 donor. After 21 days of follow up, the eyes were assessed for epitheliopathy and and mice were randomized into treatment groups. Each of the shield-treated eyes received 1 application of the shield at time 0. The topical solution treated mice were treated as follows: (loteprednol etabonate, twice a day) sodium hyaluronate, and saline, 4 times a day).

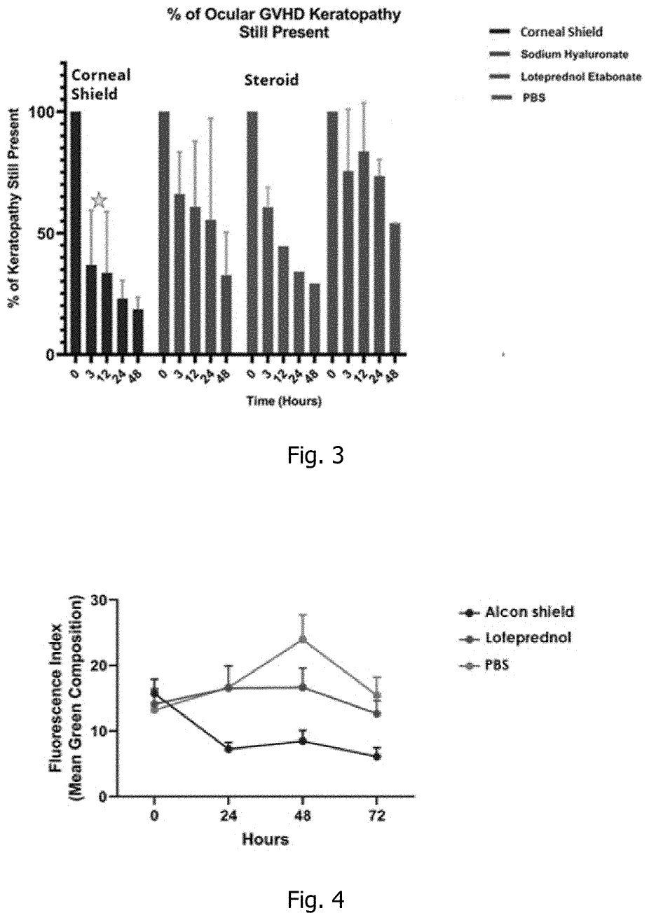

FIG. 3 illustrates that the application of only the dissolvable medical device (without a steroid) provided faster healing of the corneal damage vs. a topical steroid (2 times a day) within first 24 hours according to the percent of Keratopathy still present. The one time treatment with the corneal shield promoted faster recovery and maintenance of effect vs. other topical formulations

Mean Green Fluorescence Index test (demonstrates corneal damage) is provided as follow:

Under Isoflurane Sedation, 1 drop of Fluorescein is placed on the eye to be assessed

After 1 minute, the fluorescein is flushed with PBS

The eye is then placed under blue light and a picture is taken

Settings were unchanged throughout the experiment

The picture is then analyzed through measuring the Mean Green Fluorescence in the area of interest (Cornea)

Mean Green Fluorescein provides an objective measure of corneal epitheliopathy

FIG. 4 illustrates that the application only the dissolvable medical device (without using steroid) provides faster healing of the damage corneal vs. a topical steroid (BID) within first 24 hours according to the Fluorescein Index Change test.

Fluorescence Index test (demonstrates corneal damage) is provided as follow:

Under Isoflurane Sedation, 1 drop of Fluorescein is placed on the eye to be assessed

After 1 minute, the fluorescein is flushed with PBS

The eye is then placed under blue light and a picture is taken

Settings were unchanged throughout the experiment

The picture is then analyzed through measuring the Mean Green Fluorescence in the area of interest (Cornea)

Mean Green Fluorescein provides an objective measure of corneal epitheliopathy

1. A dissolvable medical device for placing on the outer exposed surface of the eye to heal the damage corneal wound comprising of: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers, wherein the dissolved polymeric film is not impeding visualization of ocular tissue while maintaining a protective film on outer surface of the eye.

2. The dissolvable medical device of claim 1, wherein the one or more mucoadhesive polymers are selected from the group consisting of: hyaluronic acid or salts thereof, hydroxypropylmethylcellulose (HPMC), methylcellulose, tamarind seed polysaccharide (TSP), guar, hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and combinations thereof.

3. The dissolvable medical device of claim 1 wherein the one or more mucoadhesive polymers are HP guar, hyaluronic acid, or sodium hyaluronate.

4. The dissolvable medical device of claim 3, wherein the one or more mucoadhesive polymers are present in an amount of at least 5% w/w HP guar, at least 5% w/w hyaluronic acid, at least 5% w/w polyvinyl pyrrolidine by dry weight of the polymeric film and a total amount of mucoadhesive polymers is equal to or less than 100% w/w by dry weight of the polymeric film.

5. The dissolvable medical device of claim 3, further comprising a plasticizer or softener.

6. The dissolvable medical device claim 5, wherein the plasticizer or softener is selected from the group consisting of:
polyethylene glycol (PEG), a PEG derivative, water, Vitamin E, and triethyl citrate.

7. The dissolvable medical device of claims 5, wherein the plasticizer or softener is present in an amount of from about 2% to about 30% w/w, about 5% to about 25% w/w, about 5% to about 20% w/w, or about 5% to about 15% w/w of the polymeric eye insert.

8. The dissolvable medical device of claim 6, wherein the plasticizer or softener is PEG.

9. The dissolvable medical device of claim 1, wherein the insert is comprised of approximately 40% HP guar, approximately 10% PVP, approximately 40% sodium hyaluronate, and approximately 10% PEG.

10. The dissolvable medical device of claim 1, wherein the corneal wound is selected from a group consisting of glaucoma surgery wound, vision correction laser surgery wound for treating refraction defects, cataract surgery wound and GVHD induced ocular damage in transplant patients.

11. The dissolvable medical device of claim 10, wherein the vision correction laser surgery wound for treating refraction defects is the LASIK wound or PRK wound.

12. The dissolvable medical device of claim 10, wherein the corneal wound is the glaucoma surgery wound.

13. The dissolvable medical device of claim 10, wherein the corneal damage is induced by GVHD reaction as in transplant patients.

14. The dissolvable medical device of claim 13, wherein the dissolvable medical device provides faster healing of the corneal within first 24 hours wound compared to a topical steroid treatment according to a Fluorescein Index Change test (demonstrates corneal damage).

15. A method for healing a corneal wound comprising: (a) identifying a subject in need of healing for a corneal wound selected from the group consisting of glaucoma surgery wound, cataract surgery wound, vision correction laser surgery wound and GVHD wound, and (b) placing a dissolvable medical device to the outer exposed surface of the eye to heal the corneal wound, wherein the dissolvable medical device comprising of: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers, wherein the dissolved polymeric film is not impeding visualization of ocular tissue while maintaining a protective film on outer surface of the eye.

16. The method for healing a corneal wound of claim 15, wherein the vision correction laser surgery wound is LASIK surgery wound or PRK surgery wound.
